(19) United States
(12) Patent Application Publication
(10) Pub. No.: US 2026/0199546 A1
(43) Pub. Date: Jul. 16, 2026

(54) SPRAY PATCH COMPOSITION, PROCESS FOR PREPARING IT AND KIT

(71) Applicant: FONDAZIONE ISTITUTO ITALIANO DI TECNOLOGIA, Genova (IT)

(72) Inventors: Annika Müsse, Siegen (DE); Gaia De Marzo, Monopoli (BA) (IT)

(73) Assignee: FONDAZIONE ISTITUTO ITALIANO DI TECNOLOGIA, Genova (IT)

(21) Appl. No.: 19/131,233

(22) PCT Filed: Nov. 23, 2023
(86) PCT No.: PCT/IB2023/061826

§ 371 Date: May 20, 2025

(30) Foreign Application Priority Data
Nov. 25, 2022 (IT) 102022000024417

(51) Int. Cl. A61L 26/00; C08L 1/28

(52) U.S. Cl. CPC A61L 26/0076; A61L 26/0023; A61L 26/0066; A61L 26/0071; C08L 1/28

(57) ABSTRACT

The present invention relates to a spray patch composition for the indirect real-time determination of bacterial infections on superficial wounds by means of colorimetric pH detection. In particular, said composition is a sprayable composition comprising a plant extract comprising anthocyanins, at least one film-forming polymer capable of forming a polymeric film, at least one plasticizer, at least one surfactant; and at least one solvent. The present invention also concerns a process for the production of said spray patch composition and a kit containing it.

### FIELD OF THE INVENTION

The present invention relates to a spray patch composition for the indirect real-time determination of bacterial infections on superficial wounds by means of colorimetric pH detection. In particular, said composition is a sprayable composition comprising a plant extract comprising anthocyanins, at least one film-forming polymer capable of forming a polymeric film, at least one plasticizer, at least one surfactant; and at least one solvent.

The present invention also concerns a process for the production of said spray patch composition and a kit containing it.

### STATE OF THE ART

A proper treatment of wounds is essential to allow a rapid healing process. Most wounds heal spontaneously through the cicatrization process. However, some wounds require longer healing times because they are affected by bacterial infection.

It is therefore necessary to detect the presence of a bacterial infection in order to choose the best therapeutic treatment.

In order to confirm the presence of a bacterial infection within a wound, it is usually necessary to carry out a culture examination, which takes several days before the results are available. For this reason, in order to prevent the status of the wound from worsening before the results are available, an antibiotic treatment is often prescribed on the basis of immediately accessible qualitative clues such as redness, swelling, warmth, pain and tenderness to touch, scaling and itching, as well as pustules and pus discharge. Consequently, in the absence of a diagnostic tool indicating in real time the possible presence of bacterial infections, antibiotics are often prescribed when unnecessary, as it is subsequently determined that no bacterial infection was present.

There is therefore the need to have a diagnostic tool capable of detecting bacterial infection on superficial wounds in real time.

It is known that the pH of infected skin can differ from the slightly acidic pH of skin under physiological conditions. In particular, the skin under physiological conditions has a pH of approximately 5-6, while the pH of poorly healing wounds can be as high as 10.

On the basis of differences in the pH of healthy non-wounded skin and skin with wounds and infected, several strategies for detecting the status of a wound have therefore been proposed.

In general, dressings with pH detection capabilities are often referred to as “smart” since they contain technologies that allow the monitoring of one or more wound parameters.

Most dressings comprising pH-detection systems are pre-formed patches or bandages that create quite a few problems when positioned on hard-to-reach parts of the body.

WO2012158467A2 describes a homogeneous solution or suspension useful as a coating for food preservation or medical devices or their components to detect the presence or absence of bacterial contamination.

Stephan et al., “A sprayable luminescent pH sensor and its use for wound imaging in vivo”, Exp Dermatol. 2012; 21:12:951-3 describes a method for luminescent (photographic) imaging capable of detecting pH values on tissue surfaces (e.g. wounds) and a sprayable luminescent sensor for RGB image acquisition. The luminescent sensor described herein is composed of fluorescein isothiocyanate (FITC) covalently bonded to aminoethyl cellulose (FITC-AC), and Ru(dpp)3 embedded in oxygen impermeable polyacrylonitrile (PAN) particles ((Ru(dpp)3-PAN), forming the FITC-AC/Ru(dpp)3-PAN particles. These particles are embedded in a mixture of Traumasept wound gel (Dr. August Wolff GmbH & Co. August Wolff GmbH & Co. KG, Bielefeld, Germany) and water. In order to record the luminescence, it is necessary to excite the luminophores via a 460 nm LED array mounted on a camera. The luminescence intensity of the FITC-AC particles increases with increasing pH, while the one of the Ru(dpp)3-PAN reference particles remains constant. After image acquisition, it is necessary to carry out an image transformation (white balance, RGB channel splitting), calculate the ratio of G/R luminescence intensities (provides a pH reference signal for each pixel) and construct a calibration curve.

WO2012158467A2 describes also a wound dressing composed of a polymer, a solvent and a pH indicator dye. In particular, the pH indicator dye is chosen from m-cresol purple, thymol blue, o-cresolphthalein, thymolphthalein, crystal violet, malachite green, pentamethoxy red, heptamethoxy red and hexamethoxy red. Anthocyanins are not described.

US20200069482A1 describes wound dressings that can be used to quickly and reliably determine potential healing problems of a wound. The dressings include a pH indicator as a component of a polymeric composition used to form an absorbent substrate of the dressing. The disclosed solution is in the form of a fiber, hydrogel or microspheres.

WO2008126980A1 describes pharmaceutical compositions that help to determine the status of a wound. Said compositions contain anthocyanin as an active ingredient and, if necessary, surfactants chosen from polyoxyethylene derivatives. The anthocyanin present in said sprayable composition is not retained within the composition and is destined to be released into the wound. Consequently, the anthocyanin released in the wound may come into contact with a fluid other than the fluid of interest, and result in a color change due to inadvertent contact.

The preservation of the pH indicator as a component of a polymeric composition administered to the wound is necessary to prevent the color change of the pH indicator following inadvertent contact with a fluid other than the biological fluid of interest and, consequently, to avoid an erroneous determination of the status of the wound.

It is therefore necessary to provide a diagnostic tool capable of detecting bacterial infections on superficial wounds in real time that overcomes the drawbacks of the diagnostic tools nowadays available.

In particular, there is the need to have a diagnostic tool capable of real-time detection of bacterial infections even on superficial wounds in hard-to-reach body positions.

### SUMMARY OF THE INVENTION

The Applicant has set out to overcome the technical problem relative to the lack of a diagnostic tool for determining bacterial infections that may be present in superficial wounds on hard-to-reach parts of the body.

The Applicant has advantageously made available a composition that is sprayed directly on the wound and that, by means of the spray patch mechanism, is capable of instantly forming a film that protects the wound from the external environment. Due to the presence of anthocyanins which change color in case of a basic environment of the wound and which are retained within a polymeric film, the patch obtained by spraying on the wound the composition according to the invention is capable of rapidly indicating the presence of a bacterial infection in the wound.

Therefore, a first aspect of the present invention concerns a spray patch composition comprising:
a) a plant extract comprising anthocyanins;
b) at least one film-forming polymer capable of forming a polymeric film, said film-forming polymer being chosen from ethylcellulose, carboxymethylcellulose, sodium carboxymethylcellulose, hydroxypropylcellulose, hydroxypropylmethylcellulose, cellulose acetate, starch, PVA, chitosan, polymethacrylate-based copolymers and cyanoacrylic polymers;
c) at least one plasticizer;
d) at least one surfactant; and
e) at least one solvent,
wherein said composition is in the form of a sprayable formulation, and said polymeric film is capable of retaining said anthocyanins within it.

Anthocyanins are among the most important groups of pigments in plants, and are found in flowers and fruits as well as in autumn shrubs and leaves. Therefore, anthocyanins can advantageously be extracted from various natural sources, such as red cabbage, aronia, grapes, and berries in general.

In the present invention the anthocyanins present in the spray patch composition are preferably added in the form of red cabbage extract.

The main characteristic of some of the five main elements present in the composition according to the invention is that they originate from natural and renewable sources, as described below.

In fact, the Applicant has proposed to choose environmentally friendly substances in order to have advantages both from the point of view of the safety of the formulation and from the ethical point of view.

A second aspect of the present invention relates to a process for preparing the composition as above described, comprising adding components a-d to the solvent in the order b-a-d-c.

A further aspect of the present invention is a kit comprising at least one spray container containing the composition according to the first aspect of the invention, and optionally one or more devices capable of quantifying the color change of the composition, wherein said device is preferably a smartphone.

### DETAILED DESCRIPTION OF THE INVENTION

Object of the present invention is a spray patch composition comprising:
a) a plant extract comprising anthocyanins;
b) at least one film-forming polymer capable of forming a polymeric film, said film-forming chosen from polymer being ethylcellulose, carboxymethylcellulose, sodium carboxymethylcellulose, hydroxypropylcellulose, hydroxypropylmethylcellulose, cellulose acetate, starch, PVA, chitosan, polymethacrylate-based copolymers and cyanoacrylic polymers;
c) at least one plasticizer;
d) at least one surfactant; and
e) at least one solvent,
wherein said composition is in the form of a sprayable formulation, and said polymeric film is capable of retaining said anthocyanins within it.

The composition according to the invention advantageously allows to detect in real time the presence of bacterial infections on skin surfaces affected by one or more wounds due to the color change of the anthocyanins present in the composition in basic environment.

Furthermore, the Applicant has advantageously observed that the composition according to the invention carries out multiple functions, among them, forming an impermeable film that protects the wound from friction, indicating the bacterial infection by means of a color change in case of a basic environment of the wound, and promoting the wound healing due to the antimicrobial and anti-inflammatory properties of anthocyanins.

In the present invention, the term “wound” indicates traumatic lesions of the skin characterized by the more or less extensive removal of one or more epidermal layers. In particular, wounds according to the present invention are superficial wounds.

The composition according to the invention can be sprayed directly on the wound, following the classical mechanism of the spray patch. Thanks to the presence of the film-forming polymer, said composition instantly forms a polymeric film that protects the wound from the external environment and, thanks to the presence of anthocyanins retained in the polymeric film, changes color in case of a basic environment of the wound indicating within seconds the presence of a bacterial infection.

The composition according to the invention is sprayed on at least one wound and is capable of forming a coating layer (or film) on the wound, while at the same time detecting its pH, and thus the possible presence of infection.

Therefore, in the present invention, the term “film” indicates a liquid or solid coating layer, preferably solid, which forms on a surface after application on that surface of the composition according to the invention.

The coating layer or film has a protective function of the surface on which the spray patch composition according to the invention is applied.

The composition according to the invention comprises the following five main components: a) plant extract comprising anthocyanins, b) film-forming polymer, c) plasticizer, d) surfactant and e) solvent.

Anthocyanins or anthocyans are a class of water-soluble colorants belonging to the flavonoid family. Anthocyanins are compounds that can turn from red to violet or blue as the alkalinity of the environment increases.

Anthocyanins derive from their respective aglycones, the anthocyanidins, having the following indicated formula (I):

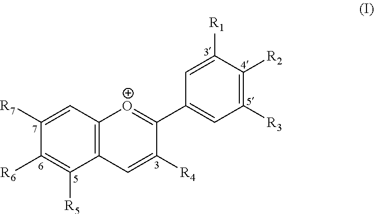

In particular, anthocyanins differ from anthocyanidins by the addition of a glycosidic group usually at position R3 and/or R4 (see Table A).

TABLE A
Anthocyanidins and their relative substituent groups

| Anthocyanidin | R₁ | R₂ | R₃ | R₄ | R₅ | R₆ | R₇ |
|---|---|---|---|---|---|---|---|
| Aurantinidine | —H | —OH | —H | —OH | —OH | —OH | —OH |
| 6-hydroxy Cyanidine | —OH | —OH | —H | —OH | —OH | —OH | —OH |
| Cyanidine | —OH | —OH | —H | —OH | —OH | —H | —OH |
| 6- hydroxy-Delfinidin | —OH | —OH | —OH | —OH | —OH | —OH | —OH |
| Delphinidin | —OH | —OH | —OH | —OH | —OH | —H | —OH |
| Europinidin | —OCH₃ | —OH | —OH | —OH | —OCH₃ | —H | —OH |
| Tricetinidin | —OH | —OH | —OH | —H | —OH | —H | —OH |
| Luteolinidin | —OH | —OH | —H | —H | —OH | —H | —OH |
| Apigeninidin | —H | —OH | —H | —H | —OH | —H | —OH |
| Pelargonidin | —H | —OH | —H | —OH | —OH | —H | —OH |
| Malvidin | —OCH₃ | —OH | —OCH₃ | —OH | —OH | —H | —OH |
| Peonidin | —OCH₃ | —OH | —H | —OH | —OH | —H | —OH |
| Petunidin | —OH | —OH | —OCH₃ | —OH | —OH | —H | —OH |
| Rosinidin | —OCH₃ | —OH | —H | —OH | —OH | —H | —OCH₃ |

The color of anthocyanins can vary from red to blue and depends on the pH of the medium in which they are and the formation of salts with heavy metals.

Therefore, anthocyanins can advantageously be used as pH indicators. In particular, the anthocyanins present in the sprayable composition will turn from red to violet or blue in case of a wound with bacterial infection, whose pH is between 6.5 and 10. Advantageously, anthocyanins are polyoxydrylated polyaromatic compounds capable of reacting with oxidants such as molecular oxygen and free radicals thus reducing the damages that these molecules can cause to cells and tissues. Thanks to their antioxidant and anti-radical activity, these substances protect plants from damages caused by ultraviolet radiations.

Therefore, in the composition according to the invention, anthocyanins not only carry out the activity of pH indicators by signaling the presence of a bacterial infection within a wound, but also the antimicrobial and anti-inflammatory activity by promoting the wound healing process.

Advantageously, the anthocyanins present in the composition are chosen from aurantinidin, 6-hydroxy-cyanidin, cyanidin, delphinidin, 6-hydroxy-delfinidin, europinidin, tricetinidin, luteolinidin, apigeninidin, pelargonidin, peonidin, petunidin, malvidin, rosinidin, and their glycosidic derivatives.

Preferably, anthocyanins are chosen from cyanidin, delphinidin, pelargonidin, peonidin, petunidin, malvidin, and their glycosidic derivatives.

Anthocyanins are preferably obtained from plant extracts, in particular from red cabbage extracts, aronia extracts, grape extracts, and in general from berry extracts of various plant species, such as blueberry, cranberry, elderberry, cloudberry, black raspberry, red raspberry, blackberry, blackcurrant, and cherry. In an even more preferred embodiment, said anthocyanins are obtained from a red cabbage extract. In fact, it has been advantageously demonstrated that the anthocyanins contained in red cabbage comprise peonidin-3-O-glucoside, petunidin-3-O-glucoside, cyanidin-3-O-glucoside, pelargonidin, cyanidin-3,5-O-diglucoside and cyanidin-3-(sinapoyl) (sinapoyl)-diglucoside-5-glucoside (Khoo et al. Anthocyanidins and anthocyanins: colored pigments as food, pharmaceutical ingredients, and the potential health benefits. Food & Nutrition Research, 61. 2017).

Therefore, in a particularly preferred embodiment, the anthocyanins present in the composition are chosen from cyanidin, pelargonidin, peonidin, petunidin, and their glycosidic derivatives, advantageously from peonidin-3-O-glucoside pethunidine-3-O-glucoside, cyanidin-3-O-glucoside, pelargonidin, cyanidin-3,5-O-diglucoside, cyanidin-3-(sinapoyl) (sinapoyl)-diglucoside-5-glucoside.

In the composition according to the invention, the plant extract comprising anthocyanins, intended as a dry extract, i.e. after removal of the extraction solvent, is present in an amount of from 0.5% to 25%, preferably from 1% to 15%, more preferably from 2% to 10%, and even more preferably from 3% to 8% by weight relative to the weight of the solvent.

The film-forming polymer present in the composition is a polymer capable of forming a film and is chosen from ethylcellulose, carboxymethylcellulose, sodium carboxymethylcellulose, hydroxypropylcellulose, hydroxypropylmethylcellulose, cellulose acetate, starch, PVA, chitosan, polymethacrylate-based copolymers, cyanoacrylic polymers.

In a particularly preferred embodiment, the film-forming polymer is ethylcellulose, a derivative of cellulose that is particularly easy to process coming from widely available renewable sources.

Furthermore, another advantage of ethylcellulose relates to its resistance to water, which can potentially ensure a long/medium term wound coverage and protection of the wound from external agents.

In the composition according to the invention, the film-forming polymer is present in an amount of from 1% to 10%, preferably from 2% to 6%, even more preferably from 2% to 4% by weight relative to the weight of the solvent.

In a particularly preferred embodiment, the film-forming polymer is present in an amount of 3% by weight relative to the weight of the solvent.

More preferably, the composition of the invention comprises ethylcellulose as a film-forming polymer in an amount of 3% by weight relative to the weight of the solvent. The Applicant has noted that compositions comprising a proportion of film-forming polymer above 10% by weight relative to the weight of solvent were too dense and unsuitable for spray use according to the invention.

The third element present in the composition according to the invention is a plasticizer chosen from glycerol, polyethylene glycol, sorbitol, xylitol, dibutylphthalate, propylene glycol, triethylcitrate and plasticizers belonging to the classes of maleates, phthalates, adipates, sebacates and citrates.

In the composition according to the invention, the plasticizer is present an amount between 10% and 80% by weight relative to the dry weight of film-forming polymer, preferably between 20% and 60% by weight relative to the dry weight of film-forming polymer, even more preferably between 25% and 40% by weight relative to the dry weight of film-forming polymer. More preferably, the composition according to the invention comprises the plasticizer in an amount of 30% by weight relative to the dry weight of film-forming polymer.

In a preferred embodiment, the plasticizer present in the composition is glycerol. Glycerol is a non-toxic compound often used to improve the mechanical properties of materials, as it reduces the fragility and increases the elastic behaviour of films. It is an essential component for obtaining a film with good mechanical properties.

In a preferred embodiment, the composition of the invention comprises ethylcellulose as a film-forming polymer and glycerol as a plasticizer.

In a particularly preferred embodiment, the composition of the invention comprises glycerol as a plasticizer in an amount of 30% by weight relative to the dry weight of ethylcellulose.

The composition of the invention comprises a surfactant whose function is to ensure that the water-soluble anthocyanin extract mixes homogeneously with the composition and that the anthocyanins are fixed within the polymeric network of the dry film (see examples 2 and 3).

According to the invention, the surfactant is a nonionic surfactant, such as, for example, ethoxylated glycols, ethoxylated fatty acids, polyoxamers, esters of glycerol with fatty acids, and esters of ethoxylated sorbitan derivatives with fatty acids.

Advantageously, the surfactant is preferably chosen from ethoxylated derivatives of sorbitan from the Tween and Span family, in particular polyoxyethylene sorbitan monolaurate (Tween 20) and polyoxyethylene sorbitan monopalmitate (Tween 40).

In a preferred embodiment, the surfactant is polyoxyethylene sorbitan monolaurate (Tween 20). Said preferred surfactant is biodegradable and approved by the US FDA as an additive for food and pharmaceutical preparations.

The nonionic surfactant is present in an amount between 0.05% and 0.5%, more preferably between 0.09% and 0.4%, even more preferably between 0.1% and 0.3% by weight relative to the weight of the solvent.

In a particularly preferred embodiment, the surfactant is polyoxyethylene sorbitan monolaurate (Tween 20) and is present in an amount of from 0.1% to 0.2% by weight relative to the weight of the solvent. In particular, the Applicant has observed that the nonionic surfactant polyoxyethylene sorbitan monolaurate (Tween 20) in an amount of 0.18% by weight relative to the weight of the solvent is capable of homogenizing the solution and keeping the anthocyanins within the polymeric network.

Finally, the composition of the invention comprises a solvent in which the previously described components a-d are solubilized.

In particular, according to the invention, it is important to use a solvent that evaporates quickly and leaves a homogeneous film.

The solvent is preferably chosen from ethanol, ethyl acetate, pentanol and isopropyl alcohol.

In a preferred embodiment, the solvent is chosen from ethanol and ethyl acetate, substances included in the list of low-risk solvents of the FDA.

In a particularly preferred embodiment, the solvent is ethyl acetate.

In the experiments conducted by the Applicant, the composition found to be the best comprises:
ethyl acetate as solvent;
red cabbage extract in an amount of 6% by weight relative to the weight of the solvent;
ethylcellulose in an amount of 3% by weight relative to the weight of the solvent;
glycerol in an amount of 30% by weight relative to the dry weight of ethylcellulose; and
polyoxyethylene (20) sorbitan monolaurate (Tween 20) in an amount of 0.18% by weight relative to the weight of the solvent.

In a further embodiment, the composition may further comprise one or more substances with antibacterial properties.

The composition according to the invention can advantageously be used as a diagnostic tool to detect in real time the possible presence of bacterial infections on one or more superficial wounds and/or in the surrounding skin.

By spraying the composition according to the invention on one or more skin surfaces affected by one or more wounds, it is possible to form a spray patch in situ having a diagnostic function for any bacterial infections present in the wound and/or surrounding skin and an antimicrobial function for wound healing.

The composition according to the invention can be obtained by adding components a-d of the composition to the solvent in the order b-a-d-c (i.e. first add the film-forming polymer (b), then the plant extract comprising anthocyanins (a), then the surfactant (d), and finally the plasticizer (c)).

As shown in the experimental section, the order of addition of the components to the solvent is essential for obtaining a functional spray patch.

Indeed, when the order of addition of components a-d to the solvent was changed, a non-homogeneous composition has been obtained in which the anthocyanins were not dispersed throughout the entire volume, but accumulated at the bottom of the beaker (example 4 and FIG. 7a).

In detail, the process comprises the steps of:
i) Solubilization of the film-forming polymer (chosen from ethylcellulose, carboxymethylcellulose, sodium carboxymethylcellulose, hydroxypropyl-cellulose, hydroxypropylmethylcellulose, cellulose acetate, starch, PVA, chitosan, polymethacrylate-based copolymers, cyanoacrylic polymers) in the solvent for 60 minutes;
ii) Addition of the plant extract comprising anthocyanins and stirring for a further 30 minutes;
iii) Addition of the surfactant and stirring for 10 minutes;
iv) Final addition of plasticizing agent and stirring for a further 10 minutes.

In a preferred embodiment, the red cabbage extract is obtained by following a “green” protocol that does not involve the use of harmful substances. In particular, red cabbage extract is obtained by following a process comprising soaking red cabbage leaves in hot deionized water (80° C.) for an hour, obtaining a liquid that is then filtered of impurities and heated on a hot plate until the extraction solvent (deionized water) has completely evaporated. Finally, a small amount of water is added to dissolve the sediment again for further use.

The percentage of extract present in the composition according to the invention defined above is based on the weight of the sediment obtained after total evaporation of the extraction solvent.

In a preferred embodiment, the process comprises adding to the ethyl acetate the following preferred components and in the following order: ethylcellulose as a film-forming polymer, the red cabbage extract comprising anthocyanins, Tween 20 as a surfactant and glycerol as a plasticizer.

Another aspect of the present invention comprises a kit comprising at least one spray container containing the composition as described above, and optionally one or more devices capable of quantifying the color change of the composition.

Thanks to the use of technical devices, the kit of the invention enables the quantification of color change visible to the naked eye, as well as detectable changes to parts of the light spectrum not visible to the naked eye.

In a preferred embodiment, said device is a smartphone.

In another embodiment, the kit also comprises one or more antibiotics to be used in case the spray patch composition according to the invention indicates the presence of bacterial infection in the wound and/or surrounding skin.

In particular, in said embodiment, the kit comprises one or more antibiotics against the main micro-organisms responsible for wound infections.

Among the main micro-organisms responsible for wound infections there are the Staphylococcus aureus, Escherichia coli, Pseudomonas aeruginosa, Proteus mirabilis, Klebsiella pneumoniae, coagulase-negative staphylococci, bacteria belonging to the genus Enterobacter, bacteria belonging to the genus Enterococcus, bacteria belonging to the D group of streptococci, other aerobic Gram-positive bacteria and fragile bacteroides.

The present invention will be further illustrated below by means of a number of preparatory examples and the figures relative to them, which are provided for purely indicative purposes and without any limitation of the present invention.

### DESCRIPTION OF FIGURES

In the following experimental part and in the description of the figures below, the percentages of red cabbage extract, ethylcellulose and Tween 20 are expressed as a weight percentage relative to the weight of the solvent, while the amount of glycerol is expressed as a weight percentage relative to the dry weight of the ethylcellulose.

FIG. 1 represents the macroscopic images of: a) the commercial spray patch described in the example 1, b) the film formed from the composition K shown in Table 1, and c) the film formed from the composition B indicated in Table 1.

In FIG. 1d) is shown a microscopic image of the commercial spray patch. In FIG. 1e) is shown a microscopic image of the film obtained from the composition K indicated in Table 1. Both FIGS. 1d) and 1e) show uniformity of polymeric aggregation.

FIG. 2 shows a) the test composition obtained by mixing, as described in the example 2, the dry red cabbage extract resuspended with composition B indicated in Table 1; b) the results of the assay carried out with one drop of the test composition placed in contact with six buffers at pH in a range between 5 and 10 to test the system's detection capability.

FIG. 3 shows a) the test composition obtained by mixing, as described in the example 2, the dry red cabbage extract resuspended with composition B indicated in Table 1, sprayed on a plastic substrate; b) resulting patch partially in contact with pH 8 buffer in the area indicated by the arrow: the color shifts to blue; and c) resulting patch placed in contact with the skin.

FIG. 4 shows a) the test composition obtained by mixing, as described in the example 2, the dry red cabbage extract resuspended with composition K indicated in Table 1, sprayed on the plastic substrate; b) resulting patch partially in contact with pH 8 buffer in two different positions of the patch.

FIG. 5 shows the spray patch formed by a) the composition 3B; b) the composition 3A, and c) the composition 3C, as described in the example 2.

FIG. 6 shows a) the spray patch resulting from the composition 4B of the example 3 sprayed on the plastic substrate b) said spray patch placed on the skin.

FIG. 7 shows the results of the experiment described in the example 4. In particular, the FIG. 7a shows the composition prepared as described in the example 4, wherein the dry red cabbage extract resuspended has been added after the Tween 20 and the FIG. 7b shows the resulting dried patch, in which the film itself is colourless.

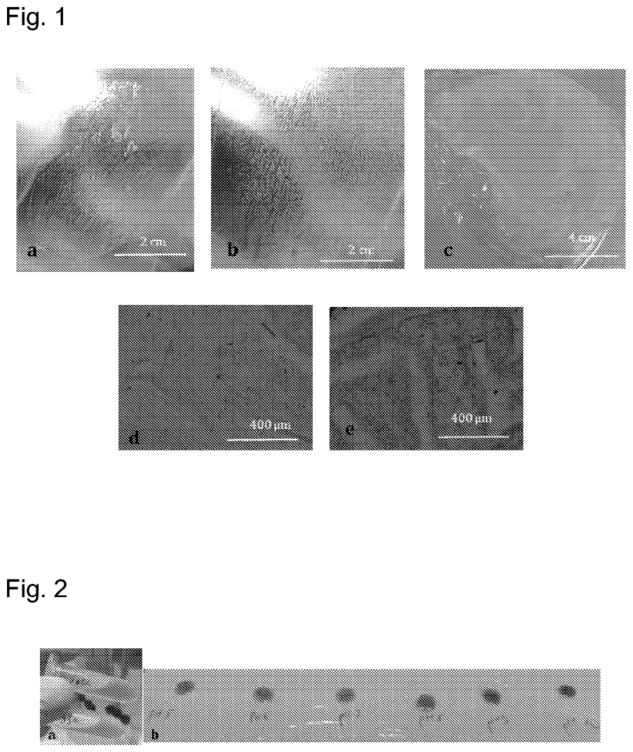

### EXAMPLES

### Example 1: Realization of Exemplary Spray Compositions A to Q

In order to assess the best composition in terms of homogeneity, sprayability and solvent evaporation rate, different compositions (A-Q shown in Table 1) of solvent, polymer and plasticizer were made. In particular, the type of solvent used and the concentrations of ethylcellulose (preferred film-forming polymer) and glycerol (preferred plasticizer) were varied.

### Homogeneity of Composition

Various concentrations of ethylcellulose (from 3% to 12% w/w) and glycerol (with percentages from 0% to 60%) were tested with the following solvents: 95% ethanol, ethyl acetate, 90% isopropanol (referred to in Table 1 as “IPA”) and pentanol.

All tested solvents are included in the FDA list of low-risk solvents.

Table 1 summarizes the compositions obtained:

TABLE 1

| Sample | Ethylcellulose (% w/w) | Glycerol (% w/w) | Solvent |
|---|---|---|---|
| A | 3 | 0 | Ethanol 95% |
| B | 3 | 30 | Ethanol 95% |
| C | 3 | 60 | Ethanol 95% |
| D | 6 | 0 | Ethanol 95% |
| E | 6 | 30 | Ethanol 95% |
| F | 6 | 60 | Ethanol 95% |
| G | 12 | 0 | Ethanol 95% |
| H | 12 | 30 | Ethanol 95% |
| I | 12 | 60 | Ethanol 95% |
| J | 3 | 0 | Ethyl acetate |
| K | 3 | 30 | Ethyl acetate |
| L | 3 | 60 | Ethyl acetate |
| M | 3 | 0 | IPA 90% |
| N | 3 | 30 | IPA 90% |
| O | 3 | 60 | IPA 90% |
| P | 3 | 0 | Pentanol |
| Q | 3 | 60 | Pentanol |

In all the cases, the ethylcellulose dissolved completely within a maximum of one hour, and it was concluded that all compositions, with the exception of compositions containing ethylcellulose 12% w/w (compositions G-I), can be used as sprayable compositions.

It was observed that compositions containing 12% w/w ethylcellulose (compositions G-I) were too dense for spray bottling.

### Sprayability of the Composition

In order to test the nebulization of the mixed components, a commercial spray bottle without propellant gas was used. In particular, it has been chosen a plastic casing made of PVC.

It was possible to spray all the compositions realized and indicated in Table 1, with the exception of the compositions containing 12% w/w ethylcellulose in ethanol (compositions G-I in Table 1).

The films obtained from the spray compositions realized were compared to the films of a commercial spray patch (Spray Patch, Solchim S.R.L.). All sprayed compositions were capable of forming a film, but the best films in terms of homogeneity and similarity to the commercial film were those obtained from B and K compositions (indicated in bold in Table 1).

### Evaporation Rate

The compositions shown in Table 1, with the exception of the G-I compositions found to be non-homogeneous, were tested in a further assay to calculate the time required to form the film.

The results are summarized in Table 2 below:

TABLE 2

| Sample | Evaporation time (min:sec) |
|---|---|
| A | 3:38 |
| B | 4:47 |
| C | 4:45 |
| D | 11:16 |
| E | 6:08 |
| F | 5:47 |
| J | 4:00 |
| K | 2:00 |
| L | 2:30 |
| M | 6:00 |
| N | 16:00 |
| O | 13:00 |
| P | 1 h+ |
| Q | 1 h+ |

B and K compositions were the best in terms of homogeneity, sprayability, film-forming ability and evaporation rate. These samples were chosen for further testing.

### Example 2: Creation of a Composition According to the Invention

In order to create the composition according to the invention, anthocyanins were extracted from red cabbage by immersing red cabbage leaves (150 g) in hot deionized water (550 ml−80° C.) for one hour, obtaining an aqueous solution which was then filtered of impurities with 0.22 μm filters. A part of the solution was put in contact with a 1M NaOH basic solution to check that the extraction protocol did not interfere with the pH detection ability of the anthocyanins.

A 10 ml portion of the solution was heated on a hot plate at 50° C. until the evaporation of the water. After the evaporation of the water, 300 mg of dry extract having the appearance of a purple sediment was obtained at the bottom of a Petri dish, comprising 1.42 mg of anthocyanins, which were taken up with small amounts of water.

In order to obtain a polymeric composition according to the invention, the dry red cabbage extract has been added to the composition B of Table 1.

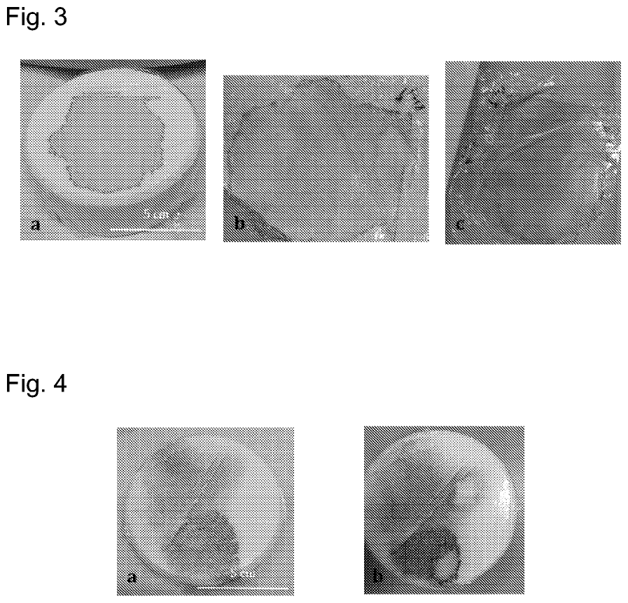

In particular, 150 mg of dry red cabbage extract resuspended in water (corresponding to about 3% of the weight of the solvent) were dissolved in 4.85 g of 95% ethanol (solvent) in which 0.145 g (corresponding to about 3% of the weight of the solvent) of ethylcellulose (film-forming polymer) had previously been dissolved. To the resulting solution have been finally added 0.0435 g (corresponding to about 30% of the weight of ethylcellulose) of glycerol (plasticizing agent). A drop of the composition has been put in contact with six different buffers at pH in a range between pH 5 and 10 for testing the detection ability of the system. In FIG. 2b it is possible to differentiate an acid solution from a basic one by color difference (pH5 is pink, pH6 is dark pink, pH7 is violet, pH 8 is blue, pH9 is dark blue, pH10 is green). The resulting polymeric composition has been sprayed on a plastic substrate where it has been formed a homogeneous and colored polymeric film (FIG. 3a). It has been applied a small quantity of pH 8 buffer for simulating the basic environment of the wound and the subsequent shifting of the color toward blue is indicated by the arrow in FIG. 3b. Using the skin as substrate, the color shifting is still visible (FIG. 3c).

The same experiment has been conducted with ethyl acetate as solvent (composition K in Table 1 comprising 150 mg of dry red cabbage extract resuspended in water (3% of solvent weight). The composition has been sprayed on the plastic substrate (FIG. 4a) and shows a color change upon contact with the solution at pH 8 (FIG. 4b).

During the experiments, the Applicant has noted that the anthocyanins were not completely attached to the polymeric film, but leaked into the buffer solution. In order to overcome this problem, three compositions similar to the composition previously described were prepared, using ethyl acetate as solvent, to each of which a different nonionic surfactant chosen from Tween 20, Tween 80 and Triton X 100 was added in an amount of 0.36% of the weight of the solvent before the addition of the plasticizer. (circa 17.46 mg).

It has been observed that the composition containing Tween 20 had a higher homogeneity, whereas the other compositions with Tween 80 and Triton-X had a lower homogeneity.

Therefore, for the composition comprising Tween 20 as surfactant, the degree of anthocyanin adhesion to the polymer and the visibility of the color change were tested. In order to do this, two compositions (3A and 3B) similar to the previous one were prepared with Tween 20, respectively with and without the addition of glycerol. A third composition (3C) has been realized with glycerol and a double amount of dry red cabbage extract (300 mg) resuspended in water equal to approximately 6% of the weight of the solvent.

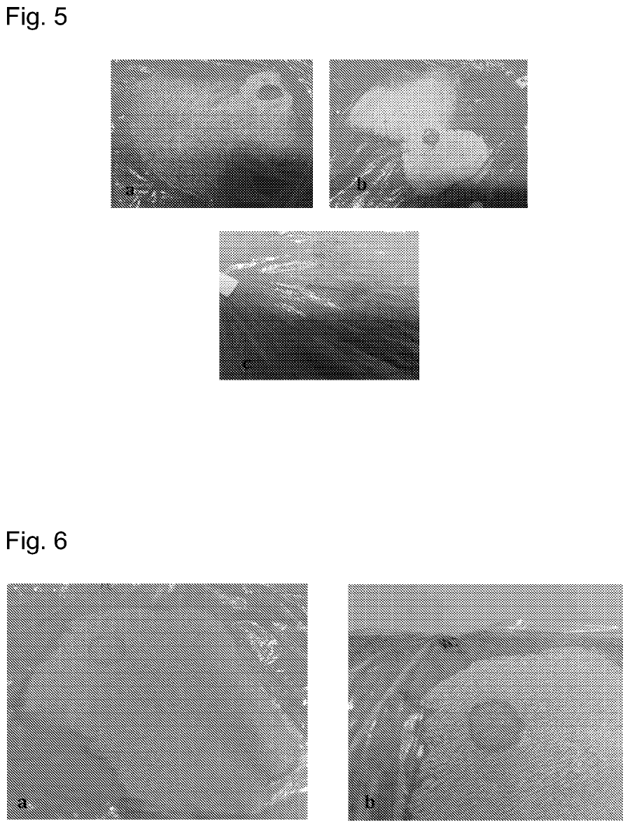

FIG. 5a shows the results obtained. By spraying the composition 3B without glycerol, a film was obtained that showed a color change after the application of the buffer at pH 8 solution and in which no anthocyanins were visible in the buffer. By spraying the composition 3A with glycerol, the result was the same (FIG. 5b), but the polymeric film showed a more pronounced color relative to the film obtained with the composition without glycerol.

Using the composition 3C comprising dry red cabbage extract resuspended in water in an amount of 6% by weight relative to the weight of the solvent, the color change was much more visible (FIG. 5c).

### Example 3: Influence of Glycerol and of Three Different Surfactant Concentrations

The Applicant has realized six compositions in 4.85 g of ethyl acetate comprising the components and quantities of the following Table 3, where all percentages are by weight relative to the weight of the solvent, except the one of the glycerol (by weight relative to the weight of the ethylcellulose).

TABLE 3

| | 4A | 4B | 4C | 4D | 4E | 4F |
|---|---|---|---|---|---|---|
| Red | 6% | 6% | 6% | 6% | 6% | 6% |
| cabbage | | | | | | |
| extract | | | | | | |
| Ethyl- | 3% | 3% | 3% | 3% | 3% | 3% |
| cellulose | | | | | | |
| Glycerol | 30% | 30% | 30% | — | — | — |
| Tween 20 | 0.09% | 0.18% | 0.36% | 0.09% | 0.18% | 0.36% |

From the present experiment, it was observed that:
all the six compositions were sprayable and, in general, the polymeric film appeared more colorful when glycerol was added to the composition; and
the best surfactant concentration was 0.18% w/w.

In fact, Tween 20 at 0.18% w/w was capable of homogenizing the composition and keep the anthocyanins within the polymeric network.

FIG. 6 shows the spray patch resulting from composition 4B, considered as the composition that provides the best result in terms of homogeneity, sprayability, film-forming ability and evaporation rate.

### Example 4: Influence of the Mixing Order of the Components on Homogeneity

The Applicant has tried to change the mixing order of the components of the composition to see what influence it had on the homogeneity of the composition, and in particular on the dispersion of the anthocyanins.

Unlike the above-described experiments, the dry red cabbage extract resuspended in water was added after Tween 20.

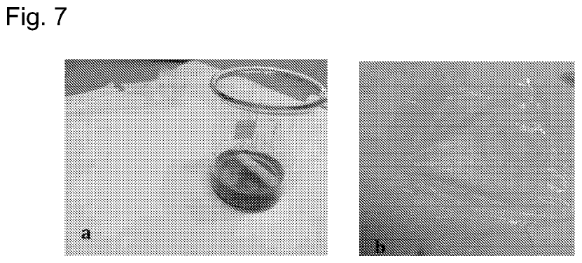

At first, ethyl acetate (solvent) was inserted into a beaker, then ethylcellulose (polymer), glycerol (plasticizer) and Tween 20 (surfactant) were directly added. After 1 hour of stirring, the red cabbage extract was added and the stirring was continued. The result was a non-homogeneous composition in which the red cabbage extract was not dissolved in the entire volume, but accumulated at the bottom of the beaker (FIG. 7a). The dried patch resulting from this composition was transparent even after placing a drop of buffer solution at pH 8 on it: no change in color was visible (FIG. 7b). In view of this, it has been demonstrated that the mixing order of the components is essential for obtaining a functional spray patch.

Therefore, the correct mixing order of the components is the following: solvent (preferably ethyl acetate), polymer (preferably ethylcellulose), red cabbage extract containing anthocyanins, surfactant (preferably Tween 20) and plasticizer (preferably glycerol).

Upon conclusion of all the carried-out experiments, the particularly preferred composition for the spray patch comprises:
ethyl acetate as solvent;
red cabbage extract in an amount of 6% by weight relative to the weight of the solvent;
ethylcellulose in an amount of 3% by weight relative to the weight of the solvent;
glycerol in an amount of 30% by weight relative to the dry weight of ethylcellulose; and
Tween 20 in an amount of 0.18% by weight relative to the weight of the solvent.

1. A spray patch composition comprising:
a) a plant extract comprising anthocyanins;
b) at least one film-forming polymer capable of forming a polymeric film, said film-forming polymer being selected from the group consisting of ethylcellulose, carboxymethylcellulose, sodium carboxymethylcellulose, hydroxypropylcellulose, hydroxypropylmethyl-cellulose, cellulose acetate, starch, PVA, chitosan, polymethacrylate-based copolymers and cyanoacrylic polymers;
c) at least one plasticizer;
d) at least one surfactant; and
e) at least one solvent,
wherein said composition is in the form of a sprayable formulation, and said polymeric film is capable of retaining said anthocyanins within it.

2. The composition according to claim 1, wherein said anthocyanins are selected from the group consisting of aurantinidin, 6-hydroxy-cyanidin, cyanidin, delphinidin, 6-hydroxy-delfinidin, europinidin, tricetinidin, luteolinidin, apigeninidin, pelargonidin, peonidin, petunidin, malvidin, rosinidin, and their glycosidic derivatives.

3. The composition according to claim 1, wherein said plant extract is selected from the group consisting of red cabbage extract, aronia extract, grape extract, and plant berry extract.

4. The composition according to claim 1, wherein said plant extract comprising anthocyanins, intended as a dry extract after removal of the extraction solvent, is present in an amount of from 0.5% to 25% by weight relative to the weight of the solvent.

5. The composition according to claim 1, wherein said at least one film-forming polymer is ethylcellulose.

6. The composition according to claim 1, wherein said film-forming polymer is in an amount between 1% and 10% by weight relative to the weight of the solvent.

7. The composition according to claim 1, wherein said at least one plasticizer is selected from the group consisting of glycerol, polyethylene glycol, sorbitol, xylitol, dibutylphthalate, propylene glycol, triethylcitrate and plasticizers belonging to the classes of maleates, phthalates, adipates, sebacates and citrates.

8. The composition according to claim 1, wherein said plasticizer is present in an amount between 10% and 80% by weight relative to the dry weight of film-forming polymer.

9. The composition according to claim 1, wherein said at least one surfactant is a nonionic surfactant.

10. The composition according to claim 1, wherein said nonionic surfactant is present in an amount between 0.05% and 0.5%.

11. The composition according to claim 1, wherein said at least one solvent is selected from the group consisting of ethanol, ethyl acetate, pentanol, isopropyl alcohol.

12. A method of detecting bacterial infection on one or more superficial wounds and/or on surrounding skin of a patient in need thereof with the composition according to claim 1, said method comprising
spraying said composition on said one or more superficial wounds and/or on said surrounding skin of said patient; and
forming a spray patch in situ, wherein said spray patch is a diagnostic tool capable of detecting bacterial infections on said one or more superficial wounds and/or on said surrounding skin of said patient.

13. A process for preparing the composition according to claim 1, comprising adding to the at least one solvent the components a-d of said composition in the order b-a-d-c.

14. A kit comprising at least one spray container containing the composition according to claim 1, and optionally one or more devices capable of quantifying the color change of the composition.

15. The kit according to claim 14, further comprising one or more antibiotics.

16. The composition according to claim 4, wherein said plant extract is present in an amount of from 1% to 15% by weight relative to the weight of the solvent.

17. The composition according to claim 6, wherein said film-forming polymer is in an amount between 2% and 6% relative to the weight of the solvent.

18. The composition according to claim 8, wherein said plasticizer is present in an amount between 20% and 60% by weight relative to the dry weight of film-forming polymer.

19. The composition according to claim 9, wherein said at least one surfactant is a nonionic surfactant selected from the group consisting of ethoxylated sorbitan derivatives of Tween and Span family.

20. The composition according to claim 10, wherein said nonionic surfactant is present in an amount between 0.09% and 0.4% by weight relative to the weight of the solvent.
